(19) United States
(12) Patent Application Publication
(10) Pub. No.: US 2022/0240758 A1
(43) Pub. Date: Aug. 4, 2022

(54) TREATMENT INSTRUMENT FOR ENDOSCOPE

(71) Applicant: Ovesco Endoscopy AG, Tübingen (DE)

(72) Inventors: Nico Hofmann, Wernau (DE); Gabor Conrad, Dettenhausen (DE); Alexander Meining, Würzburg (DE); Chi-Nghia Ho, Reutlingen (DE)

(73) Assignee: Ovesco Endoscopy AG, Tübingen (DE)

(21) Appl. No.: 17/623,799

(22) PCT Filed: Jul. 2, 2020
(86) PCT No.: PCT/EP2020/068720

§ 371 Date: Dec. 29, 2021

(30) Foreign Application Priority Data
Jul. 4, 2019 (DE) 10 2019 118 043.0

(51) Int. Cl. A61B 1/00; A61B 1/018; A61B 10/06

(52) U.S. Cl. CPC A61B 1/00087; A61B 1/00101; A61B 2017/2939; A61B 10/06; A61B 1/018

(57) ABSTRACT

A treatment instrument which can be coupled to a distal end of an endoscope and has a hollow body and a gripping element. The gripping element can be operated via an actuation mechanism situated inside the hollow body. The gripping element is configured in the form of a cap with which a distal end of the hollow body can be closed. The invention also relates to an endoscope having a distal end which is coupled or can be coupled to a treatment instrument according to the invention.

### TECHNICAL FIELD

The present invention relates to a treatment instrument/smart function carrier/retrofit kit which can be coupled to (e.g., attached to the outer circumferential side of) a distal end/end portion/endoscope head of an endoscope and has a hollow body (coupling portion) and a gripping element (actuator portion), wherein the gripping element (actuator portion) can be operated via an actuation mechanism situated inside the hollow body (coupling portion). Moreover, the present invention relates to an endoscope having a distal optical system and, if necessary, a working channel as well as having a distal end/end portion/endoscope head which is coupled to the treatment instrument.

### BACKGROUND ART

A generic treatment instrument/smart function carrier/retrofit kit is known, for example, from EP 1 543 786 B1. Said treatment instrument has a cap-like or sleeve-like proximal attachment portion (facing the attending physician) and a distal actuator portion (facing away from the attending physician) which can be operated using operating wires. When examining human bodies by means of an endoscope equipped/retrofitted with the generic treatment instrument, it has turned out, however, that a distal optical system of the endoscope at the endoscope head is particularly susceptible to soiling which may impair the view of the attending physician (an operating surgeon or operator).

### SUMMARY OF THE INVENTION

Applicant found that this tendency to soiling results from the fact that dirt on the optical system cannot be sufficiently removed/rinsed by a cleaning device/rinsing nozzle common for endoscopes any more, as it collects inside the sleeve-like coupling portion.

With this in mind, it is an object of the present disclosure to provide a treatment device/smart function carrier/retrofit kit of the afore-mentioned species for an endoscope and, respectively, for adaptive/selective mounting on/to an endoscope which reduces the probability of soiling of the (distal) optical system of the endoscope restricting the operator's view.

This object is achieved by a treatment instrument or treatment device/smart function carrier/retrofit kit comprising the features of claim 1 and by an endoscope (including optical system and working channel, where necessary) comprising the features of claim 14 or 15. Advantageous developments of the treatment instrument are the subject matter of the subclaims.

A treatment device/smart function carrier/retrofit kit according to the disclosure can be coupled to a distal end/end portion/endoscope head of an endoscope (including optical system and internal working channel within the endoscope shaft, where necessary) and comprises a hollow body/coupling portion (sleeve) as well as a gripping element/actuator portion/actuator (preferably consisting of two gripper or forceps branches). The gripping element can be operated via an actuation mechanism (operating wires). A distal end or a distal end portion of the actuation mechanism is situated inside the hollow body. In particular, the distal end/end portion of the actuation mechanism is situated inside a recess in a (circumferential) outer wall of the hollow body. The actuation mechanism or a proximal portion of the actuation mechanism extends from the hollow body to a proximal direction (facing the attending physician).

In other words, the treatment instrument according to the invention comprises, as the hollow body, a mounting member which has, at a proximal end/end portion of the treatment instrument, a mounting/attachment portion which is suited and provided for receiving a distal end/end portion/endoscope head of an inserting portion (shaft) of an endoscope to mount the treatment instrument on the distal end/end portion of the inserting portion/shaft of the endoscope. The gripping element serves as a treatment member so that tissue possibly disposed distally from the mounting portion or an object situated distally from the mounting portion, such as a foreign matter or bolus, can be treated or gripped by adjusting the treatment member or the gripping element. For causing the gripping element to be adjusted, the actuation mechanism, in particular a distal end/end portion of the actuation mechanism, is directly or indirectly connected to the gripping element, particularly to a proximal end/end portion of the gripping element.

Expressed in constructive terms, the treatment instrument according to the present disclosure comprises
a sleeve (base body) that is provided and configured at its proximal end portion (coupling portion) to be attached/fitted to/onto an endoscope shaft or endoscope head,
a gripping element, such as, e.g., two gripper or forceps branches, (pivotally) supported on a distal end portion (actuator portion) of the sleeve, and
operating wires which are mounted to the gripping element (gripper or forceps branches) (and preferably extend through the sleeve) and via which at least closing/gripping forces (and preferably also opening forces) can be transferred/applied to the gripping element (gripper or forceps branches) to appropriately operate the latter.

The treatment instrument according to the disclosure excels especially by the fact that the gripping element (in the closed position/gripping position) is in the form of a cap or flap or is configured as a cap shape (i.e. the gripper or forceps branches are shaped and dimensioned preferably in shell or flap type so that, in the gripping position, they form a preferably semi-spherical cap or flap closed at least in the distal direction) which is dimensioned so that it closes a distal end of the hollow body (open distal end face of the sleeve).

In this context, by the term “closed” a condition is understood in which a structure or, in the present case, the gripping element is at least largely impermeable at least to tissue particles, in particular to tissue particles having a minimum outer diameter of 1.1 mm. In other words, the gripping element is configured so that, in a closed position (gripping position), it at least comprises no recess having a maximum opening width of 1.1 mm or more and/or at least no gap having a maximum gap width of 1.1 mm or more.

In particular, by “closed” a condition is understood in which the gripping element of the treatment instrument coupled to a distal end/end portion of an endoscope in a closed position (gripping position) covers a lens of an optical system of the endoscope disposed at the distal end of the endoscope in a distal direction.

In other words, the treatment instrument excels by being configured to close at least largely, preferably completely, a distal end face of the hollow body and, respectively, of the mounting portion in a closed position of the gripping element in the distal direction.

By the term “gripping position” the structural position is understood which the mechanism of the gripping element or the actuation mechanism (structurally) maximally admits. I.e., in the case of gripping forceps or forceps as a gripping element, for example, the gripping position is structurally defined as the maximum position (designed position) in which the gripper or forceps branches abut against each other or are spaced apart from each other with a minimum gap by means of a possibly provided stop (in the gripping element or in the actuation mechanism). The same preferably applies also to the release position which relates/can relate to the structural position (designed position) which is admitted by the mechanism of the gripping element or by the actuation mechanism via a stop, for example, wherein it shall be pointed out in this context that in particular the release position may also be undefined (i.e., at will).

The design of the gripping element as a cap/flap may advantageously reduce the probability of soiling of an optical system of an endoscope equipped with the treatment instrument according to the invention.

The gripping element may be configured to be transparent or may be translucent at least in portions. In particular, the gripping element may be configured to be transparent or translucent at least in portions with respect to light of the spectrum visible to human beings (light of a wavelength ranging from 420 to 750 nm). Alternatively, or additionally, the gripping element can be configured to be translucent at least in portions also to infrared or ultraviolet light.

A gripping element that is configured to be transparent at least in portions advantageously enables an optical system of an endoscope equipped with a treatment instrument according to the invention to be at least partially serviceable even when the optical system is closed by the gripping element.

The gripping element (i.e., the gripper/forceps branches) may have a flat plate portion or a single-curved shell portion at one end face (or is/are configured as a flat plate portion or shell portion) which is arranged, in a closed position of the gripping element, on a distal side of the gripping element. As an alternative, the gripping element may comprise, at one end face, a shell portion conically widening toward the hollow body and, respectively, in the proximal direction, the shell portion being disposed, in a closed position of the gripping element, on a distal side of the gripping element. In particular, the flat plate portion or the (three-dimensionally curved) shell portion extends perpendicularly to a longitudinal extension axis of the hollow body. In particular, the gripping element is configured so that the flat plate portion or the single-curved shell portion or the shell portion conically widening in the proximal direction is transparent at least in portions.

By providing the flat plate portion or the single-curved shell portion, the probability of tissue damage can be advantageously reduced, for example, when an endoscope equipped with the treatment instrument encounters a stricture during examination. By providing the shell portion conically widening in the proximal direction, a stricture can be gently widened when the treatment instrument passes the same. This can be assisted by opening the gripping instrument while the treatment instrument is passing. If the flat plate portion or the single-curved shell portion is formed to be transparent at least in portions, the simple geometry makes it easier to account for any optical distortions that may occur. If the shell portion widening in the proximal direction is transparent, approaching a stricture to be passed can be facilitated.

A portion of an edge of a proximal end of the gripping element can be pivotally connected about a pivot axis via a joint having a portion of an edge of a distal end of the hollow body such that the pivot axis extends tangentially to both portions. In particular, the gripping element is configured to be shell-shaped so that the proximal end of the gripping element in the closed position of the gripping element merges flush into the distal end of the hollow body. In particular, the joint connecting the hollow body to the gripping element may be provided at the hollow body in the form of an extension protruding in the distal direction.

In other words, the opposed gripper or forceps branches of the gripping element are hinged to or close to the distal front edge of the hollow body/sleeve such that the gripper or forceps branches in the gripping position (closing position) are largely flush (without any major projections) with the circumferential surface of the hollow body/sleeve. In this way, in the gripping position of the spherical shell-type gripper or forceps branches quasi a type of cupola or dome results which is quasi adjacent to the distal front edge of the hollow body/sleeve along its circumference with the smallest possible axial gap width, without protruding beyond or retracting from the outer circumferential surface. It is pointed out that by the term “adjacent” also a defined axial space is to be understood which is not very important regarding a possible circumferential penetration of dirt or liquid.

The tangential arrangement of the joint/hinge which connects the hollow body to the gripping element helps advantageously configure the gripping element such that, in an opened position, it releases an end face of the hollow body completely or almost completely in the distal direction.

The actuation mechanism can comprise at least one actuator movably supported in the hollow body which actuator is connected to the gripping element via at least one connecting rod or at least one push rod or at least one flexible element, for example. In other words, the actuation mechanism may comprise an actuator (slide) that is movable or supported in a translational or predominantly translational manner with respect to the hollow body, the translational movement of the actuator causing a rotational movement of the gripping element or of a part or of parts of the gripping element via one or more connecting rods and/or via one or more push rods and/or via one or more flexible elements. The connecting rod(s) and/or the push rod(s) may be pivotally connected to the gripping element and/or to a part of the gripping element (i.e., one or both of the gripper/forceps branches) via a swivel/hinge joint. The connecting rod and/or the push rod may be pivotally connected to the actuator via a swivel joint. The flexible element may be tightly connected to each of the gripping element and the actuator.

When the actuator is moved toward the distal end of the hollow body, the gripping element opens. Conversely, the gripping element closes when the actuator is moved toward the proximal end of the hollow body.

The flexible element is flexible insofar as it is reversibly deformed when the actuator is displaced so that it allows a transmission of force or a transmission of movement from the actuator to the gripping element.

The connecting rod can be pivotally connected, via a joint, to a further portion (with respect to the portion provided with the joint connecting the gripping element or a gripping part to the hollow body) of the edge of the proximal end of the, particularly shell-type, gripping element or gripping part about a pivot axis such that the pivot axis is perpendicular to the further portion. In particular, the pivot axis of the joint connecting the hollow body to the gripping element or gripping part can extend in parallel to the pivot axis of the joint connecting the gripping element or gripping part to the connecting rod. Particularly, the joint connecting the hollow body to the gripping element or gripping part and the joint connecting the gripping element or gripping part to the connecting rod may be arranged on two different, particularly opposite, sides of the gripping element or gripping part.

This arrangement of the connecting rod with respect to the gripping element or gripping part makes it advantageously possible to increase an interior delimited by the hollow body and the closed gripping element.

The gripping element may comprise two gripping parts (i.e., the gripper/forceps branches). One of the two gripping parts may be firmly connected to the hollow body. The other of the two gripping parts may be pivotally or slidably connected to the hollow body. In other words, the movable gripping part may be connected to the hollow body via a swivel joint or via a slider joint.

The at least one connecting rod and/or the at least one push rod or the at least one flexible element can connect the gripping part that is movably connected to the hollow body to the actuator. In particular, a further actuator and a further connecting rod and/or a further push rod or a further flexible element may be provided, wherein the further connecting rod and/or the further push rod or the further flexible element connects the gripping part that is movably connected to the hollow body to the further actuator.

A distal end of the actuator and a distal end of the further actuator may be arranged particularly on two opposite sides of the hollow body.

Alternatively, the gripping element may comprise two gripping parts both of which are pivotally or slidably connected to the hollow body. In other words, each of the movable gripping parts may be connected to the hollow body via a swivel joint or via a slider joint.

The connecting rod and/or the push rod or the flexible element may connect the actuator to one of the two gripping parts. Another connecting rod and/or another push rod or another flexible element may be provided to connect the actuator also to the other of the two gripping parts. In particular, the treatment instrument can also comprise a further actuator which is connected to each of the two movable gripping parts via another connecting rod and/or another push rod or another flexible element. Just as in the configuration where only one gripping part is movably connected to the hollow body, also in the configuration in which both gripping parts are movably connected to the hollow body, a distal end of the actuator and a distal end of the further actuator can be disposed on two opposite sides of the hollow body.

The actuator and/or the further actuator and, respectively, the distal end of the actuator and/or the distal end of the further actuator may extend symmetrically to/at/in a plane extending centrally between two gripping parts of the gripping element. In other words, the gripping element may be configured in the way of a clamshell grab of an excavator.

In particular, the two gripping parts movably connected to the hollow body can be arranged opposite to each other such that the actuator and/or the further actuator and, respectively, the distal end of the actuator and/or the distal end of the further actuator extend(s) centrally between the two gripping parts.

If only one of two gripping parts is pivotally connected to the hollow body, the manufacture of the treatment instrument can be kept relatively simple. Providing two gripping parts which are pivotally connected to the hollow body can be of advantage insofar as the treatment instrument can be configured such that tissue which is intended to be gripped by the treatment instrument can be better visible prior to gripping.

In accordance with one aspect of the invention, the treatment instrument or the gripping element can comprise more than two gripping parts. All gripping parts can be pivotally or slidably connected to the hollow body. In other words, all gripping parts can be connected to the hollow body via at least one swivel joint or at least one slider joint. As an alternative, it is also possible to configure at least one of the gripping parts of the treatment instrument with more than two gripping parts so that it is tightly connected to the hollow body.

The treatment instrument with more than two gripping parts can be configured such that each of the gripping parts pivotally or slidably connected to the hollow body can be driven via a separately provided actuator. In other words, the treatment instrument may comprise as many actuators as gripping parts pivotally or slidably connected to the hollow body. Accordingly, each actuator can be connected to the corresponding gripping part via a (separate) connecting rod and, respectively, a (separate) push rod or a (separate) flexible element. Alternatively, it is also possible to configure the treatment instrument so that several of the gripping parts pivotally or slidably connected to the hollow body can be driven using a joint actuator. In other words, the joint actuator may be connected to several gripping parts by means of several connecting rods and, respectively, push rods or several flexible elements so that an adjustment of several gripping parts is caused by a shift of the joint actuator. It is also possible to design the treatment instrument having more than two gripping parts with several joint actuators, i.e., with several actuators by each of which several gripping parts can be driven.

The distal ends of the actuators can be arranged, in accordance with the number and the arrangement of the gripping parts, in the sides of the hollow body. In particular, the arrangement of the distal ends of the actuators can be designed so that the connecting rods and, respectively, the push rods or the flexible elements connect each of the gripping parts movably connected to the hollow body to the actuator centrally with respect to the respective gripping part. This type of arrangement is desirable in that it results in no forces, or only minor forces, in addition to the forces that enable the gripping part or gripping parts to move, which can cause twisting in the joint(s).

The gripping element or at least one of the gripping parts can have a recess with a maximum opening width of 1.1 mm or less. The recess can be particularly arranged on the gripping element such that it is located, in the closed position of the gripping element, on the distal side or on the end face of the gripping element in a central area. In particular, the recess can be configured to extend at equal or different parts in the gripping parts forming the gripping element. The recess particularly extends, in the preferred embodiment having two gripping parts, partly in the one of the two gripping parts and partly in the other of the two gripping parts. In particular, the recess may be provided in the form of a round or circular-cylindrical opening.

It is advantageously possible, by providing a recess with a maximum opening width of 1.1 mm or less, to slide a guide wire through the gripping element provided in the closed position. By limiting the extent of the recess, the gripping element can further advantageously be prevented from penetrating disturbing tissue.

An endoscope according to the invention comprises a distal end that is coupled to a treatment instrument according to the invention. A proximal portion of the actuation mechanism of the treatment instrument is guided along an outer face of the endoscope.

An alternative or additional aspect of the present invention relates to an endoscope having a distal end which comprises a lens of an optical system at an end face and which can be coupled to a treatment instrument. The treatment instrument comprises a hollow body and a gripping element. The gripping element can be operated via an actuation mechanism which has a distal end situated inside the hollow body. The actuation mechanism extends from the hollow body in a proximal direction and comprises a proximal portion which can be guided along an outer face of the endoscope.

The gripping element is configured in the form of a cap that can be moved to a closed position. The gripping element is configured so that the gripping element or a part of the gripping element, in the closed position of the gripping element, covers the lens of the optical system of the endoscope in the distal direction, when the treatment instrument is coupled to the distal end of the endoscope.

Thus, the functionality of the gripping element can advantageously be broadened, as it can be used not only for gripping but also for covering the lens.

The endoscope can comprise a rinsing channel through which rinsing liquid can be conveyed to a rinsing channel outlet opening disposed at the end face of the endoscope. The gripping element can be configured so that the gripping element or a part of the gripping element, in the closed position of the gripping element, covers the rinsing channel outlet opening at least partially in the distal direction, when the treatment instrument is coupled to the distal end of the endoscope. Moreover, the gripping element can be configured so that an inner surface of the gripping element facing in the proximal direction is spaced apart, in the closed position of the gripping element, from the lens of the optical system and from the rinsing channel outlet surface such that, in a condition in which the treatment instrument is coupled to the distal end of the endoscope, in which the gripping element is closed and in which rinsing liquid is conveyed to the rinsing channel outlet opening, rinsing liquid, i.e., at least part of the rinsing liquid conveyed to the rinsing channel outlet opening, after exiting the rinsing channel outlet opening impinges on the inner surface of the gripping element facing in the proximal direction and is reflected to the lens of the optical system.

Thus, rinsing liquid can be used not only for rinsing tissue disposed distally from the endoscope, but also for cleaning the lens of the optical system of the endoscope.

### BRIEF DESCRIPTION OF THE DRAWINGS

In the following, the present invention will be described in more detail by way of preferred embodiments with reference to the attached drawings, wherein:

FIG. 1 shows a perspective view of a first embodiment of a treatment instrument according to the invention in a condition attached to an endoscope in an opened position;

FIG. 2 shows a perspective view of a distal end of the treatment instrument shown in FIG. 1 according to the first embodiment in a closed position;

FIG. 3 shows a longitudinal section view of the distal end of the treatment instrument shown in FIG. 2 according to the first embodiment in the closed position in accordance with the sectional plane III shown in FIG. 2;

FIG. 4 shows a longitudinal section view of the distal end of the treatment instrument shown in FIG. 1 according to the first embodiment in the opened position in accordance with the sectional plane IV shown in FIG. 1;

FIG. 5 shows a frontal view of the treatment instrument shown in FIG. 2 according to the first embodiment in the closed position in accordance with the arrow V shown in FIG. 2;

FIG. 6 shows a frontal view of the treatment instrument shown in FIG. 1 according to the first embodiment in the opened position in accordance with the arrow VI shown in FIG. 1;

FIG. 7 shows a rear view of the distal end of the treatment instrument according to the first embodiment in the closed position;

FIG. 8 shows a rear view of the distal end of the treatment instrument according to the first embodiment in the opened position;

FIG. 9 shows a perspective view of a treatment instrument according to the invention in accordance with a second embodiment in a condition attached to an endoscope in an opened position;

FIG. 10 shows a longitudinal section view corresponding to FIG. 4 of a treatment instrument according to the invention in accordance with a third embodiment;

FIG. 11 shows a perspective view of a treatment instrument according to the invention in accordance with a fourth embodiment in a closed position;

FIG. 12 shows a perspective view of the treatment instrument shown in FIG. 11 in accordance with the fourth embodiment in an opened position;

FIG. 13 shows a schematic longitudinal section view of a treatment instrument according to the invention;

FIG. 14A und FIG. 14B show schematic cross-sectional views for illustration of different configurations of an actuation mechanism in accordance with the first and second embodiments;

FIG. 14C shows a schematic cross-sectional view corresponding to FIG. 14A and FIG. 14B of a treatment instrument according to the invention in accordance with a fifth embodiment with an alternative configuration of the actuation mechanism;

FIG. 15 shows a schematic longitudinal section view corresponding to FIG. 13 of a treatment instrument according to the invention in accordance with a sixth embodiment with a further alternative configuration of the actuation mechanism;

FIG. 16 shows a schematic cross-sectional view corresponding to FIG. 14A, FIG. 14B und FIG. 14C of the treatment instrument in accordance with the sixth embodiment;

FIG. 17 shows a schematic longitudinal section view corresponding to FIG. 13 of a treatment instrument according to the invention;

FIG. 18A shows a schematic cross-sectional view corresponding to FIG. 14A, FIG. 14B and FIG. 14C of the treatment instrument in accordance with a seventh embodiment;

FIG. 19 shows a schematic longitudinal section view corresponding to FIG. 17 of a treatment instrument according to the invention in accordance with an eight embodiment;

FIG. 20 shows a cross-sectional view corresponding to FIG. 18A and FIG. 18B of the treatment instrument in accordance with the eighth embodiment;

FIG. 21 shows a longitudinal section view corresponding to FIG. 3 of a treatment instrument according to the invention in accordance with a ninth embodiment in a closed position;

FIG. 22 shows a longitudinal section view corresponding to FIG. 4 of the treatment instrument in accordance with the ninth embodiment in an opened position;

FIG. 23 shows a longitudinal section view corresponding to FIG. 3 of a treatment instrument according to the invention in accordance with a tenth embodiment in a closed position;

FIG. 24 shows a longitudinal section view corresponding to FIG. 4 of the treatment instrument in accordance with the tenth embodiment in an opened position;

FIG. 25 shows a longitudinal section view corresponding to FIG. 3 of a treatment instrument according to the invention in accordance with an eleventh embodiment in a closed position;

FIG. 26 shows a longitudinal section view corresponding to FIG. 4 of the treatment instrument in accordance with the eleventh embodiment in an opened position;

FIG. 27 shows a frontal view corresponding to FIG. 5 of the treatment instrument in accordance with the eleventh embodiment in the closed position;

FIG. 28 shows a rear view corresponding to FIG. 7 of the treatment instrument in accordance with the eleventh embodiment in the closed position;

FIG. 29 shows a longitudinal section view corresponding to FIG. 3 of a treatment instrument according to the invention in accordance with a twelfth embodiment in a closed position; and

FIG. 30 shows a longitudinal section view corresponding to FIG. 4 of the treatment instrument in accordance with the twelfth embodiment in an opened position.

Equal or functionally equivalent features are provided with the same reference numerals in the individual figures.

### DETAILED DESCRIPTION OF PREFERRED EMBODIMENTS

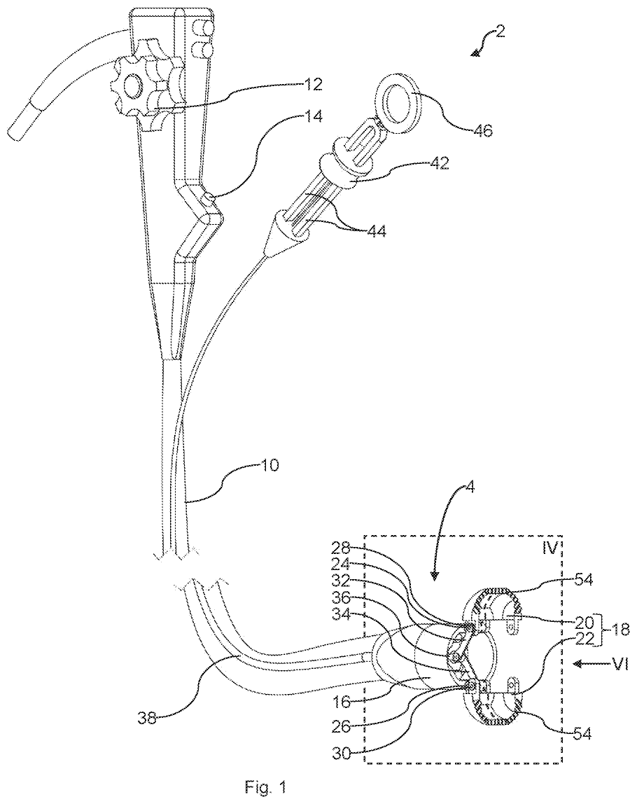

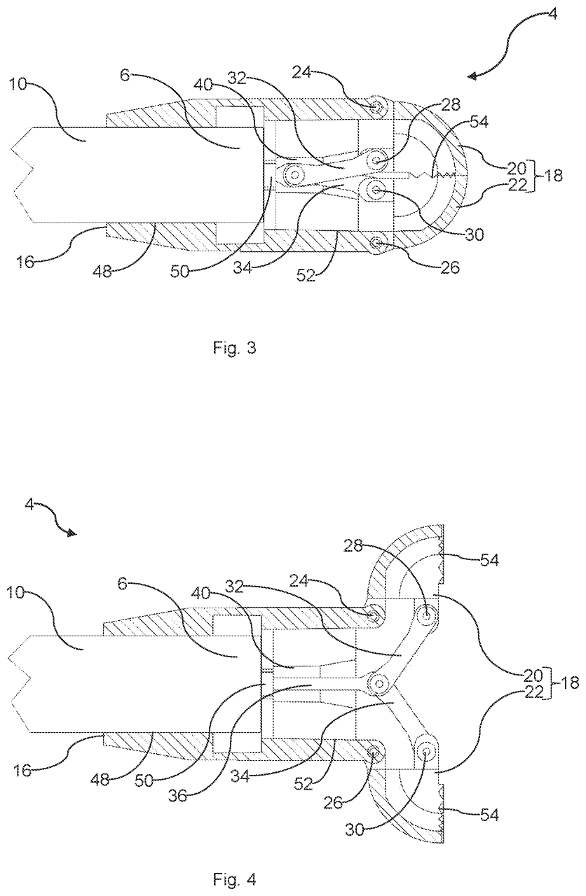

On principle, the invention relates to a treatment instrument 4 shown in FIG. 1 for retrofitting a (commercially available) endoscope 2 of the flexible shaft design. Endoscopes 2 of this type are sufficiently known from the state of the art and, therefore, will be described only superficially in the following. Usually, such endoscope 2 comprises an endoscope head 6 (see FIG. 3, for example) in which various functional units 8 not detailed in the following, such as an outlet of a primary or endoscope-internal working channel, an optical system for imaging, lamps and/or a rinsing channel outlet opening of a rinsing channel, are disposed (see FIG. 6). Furthermore, such endoscope 2 usually comprises a flexible shaft 10 comprising passively rigid portions and, preferably, an actively bendable portion (deflecting), as well as a handle 12 comprising various operating elements and terminals for connecting the various functional units 8 to a base station or the like. In particular, an inlet 14 of the primary working channel is provided on the handle 12.

The treatment instrument 4 comprises a hollow body 16 at the distal end of which a gripping element 18 including two opposite gripping parts 20 and 22 is provided. Each of the two gripping parts 20 and 22 is pivotally connected via a joint 24 and/or 26 to a corresponding portion at a distal edge of the hollow body 16.

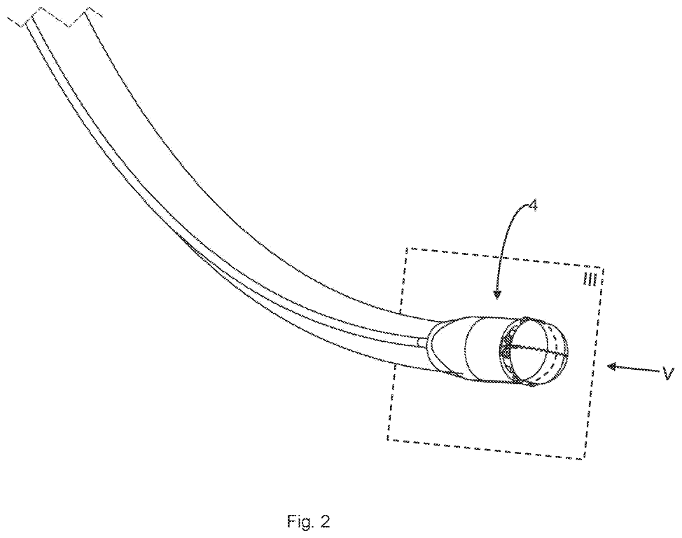

For being adjustable from an open or opened position shown in FIG. 1 to a closed position shown in FIG. 2, each of the two gripping parts 20 and 22 is connected via a respective joint 28 and 30 to a corresponding connecting rod 32 and 34, respectively. The two connecting rods 32 and 34 in turn are connected to a distal end of an actuator 36. Preferably, the actuator 36 is configured so that a rigid element forms the distal end of the actuator 36 and the rigid element is connected to a pull-push wire that extends from the rigid element in the proximal direction. In such configuration, consequently, the rigid element and the pull-push wire jointly form the actuator 36.

A tube 38 extends from a proximal end of the hollow body 16 in the proximal direction. Instead of said tube 38, alternatively a flexible wire spiral including a coating may be provided. A secondary endoscope-external working channel 40 extends from a distal end of the hollow body 16 through an external wall of the hollow body 16 to the proximal end of the hollow body 16 as well as from the proximal end of the hollow body 16 through the tube 38 to a proximal end of the tube 38. In said secondary working channel 40, the actuator 36 and, respectively, the rigid element as well as the pull-push wire of the actuator 36 is/are movably supported.

In order to allow easy shifting of the actuator 36, a slide 42 is provided at the proximal end of the actuator 36 and/or at the proximal end of the pull-push wire, said slide being movably connected to two guide rails 44 being in parallel to each other and extending from the proximal end of the tube 38 in the proximal direction. At proximal ends of the guide rails 44, a ring 46 is provided such that the ends of the guide rails 44 are connected to each other and the slide 42 is thus caught in the guide rails 44. In order to shift the actuator 36, a person using the treatment instrument 4 can put the thumb of either of his/her hands into the ring 46, can clamp the slide 42 between the index finger and the middle finger of the same hand and can adjust the slide 42 relative to the thumb by moving the index finger and the middle finger.

In order to be connectable to the endoscope head 6, the hollow body 16 comprises at a proximal end a substantially circular-cylindrical recess 48 into which the endoscope head can be inserted with a press-fit (see FIG. 3 and FIG. 4). As an alternative, or in addition to this, clip fasteners could also be provided on the hollow body 16 for connection to an endoscope head.

At a distal end of the recess 48 three stops 50 are provided, evenly spread over the inner circumference of the hollow body 16 which delimit the maximum immersion depth of the endoscope head 6. Alternatively, the stops 50 may be spread unevenly over the inner circumference of the hollow body 16.

Distally from the stops 50, the hollow body 16 has a substantially circular-cylindrical recess 52 extending to the distal end of the hollow body 16 whose inner diameter is larger than an inner diameter of the recess 48 at the proximal end of the hollow body 16. As an alternative, the inner diameters of the recesses 48 and 52 may also be equal, or the inner diameter of the recess 48 may be larger than the inner diameter of the recess 52.

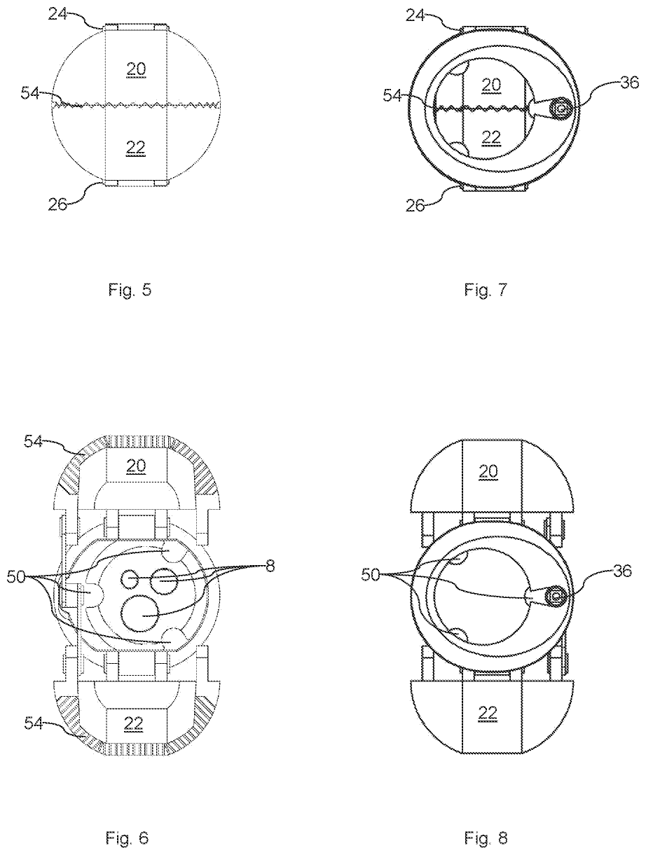

The two gripping parts 20 and 22 are configured in shell or blade shape so that, in the closed position as shown in FIG. 3, FIG. 5 and FIG. 7, together they may form a hood-shaped structure that completely closes the hollow body 16 in the distal direction. When the treatment instrument 4 according to the invention is attached to an endoscope head 6, it is thus possible to screen the endoscope head 6 and/or a lens of an optical system of the endoscope 6 from impurities by the gripping parts 20 and 22 in the closed position.

Each of the two gripping parts 20 and 22 comprises a gripping strip 54. The two gripping strips 54 have projections and recesses. The gripping strips 54 are complementary so that, in the closed position of the gripping parts 20 and 22, the projections of the gripping strip 54 of the gripping part 20 can immerse into the recesses of the gripping strip 54 of the gripping part 22, and the projections of the gripping strip 54 of the gripping part 22 can immerse into the recesses of the gripping strip 54 of the gripping part 20. Alternatively, the gripping strips 54 may also have a flat design and may be matched to each other so that the gripping strips 54 are in flush contact with each other in the closed position of the gripping parts 20 and 22.

The joints 24 and 26 which connect the gripping parts 20 and 22 to the hollow body 16 are configured such that the swivel axes thereof extend tangentially to a circumference of the hollow body 16. It is thus possible to fold the gripping parts 20 and 22 outwards such that, in the opened position as shown in FIG. 4, FIG. 6 and FIG. 8, they completely release a distal side of the recess 52.

The first embodiment shown in FIGS. 1 to 8 of the treatment instrument 4 according to the invention comprises only one actuator 36 and, correspondingly, only one endoscope-external working channel 40.

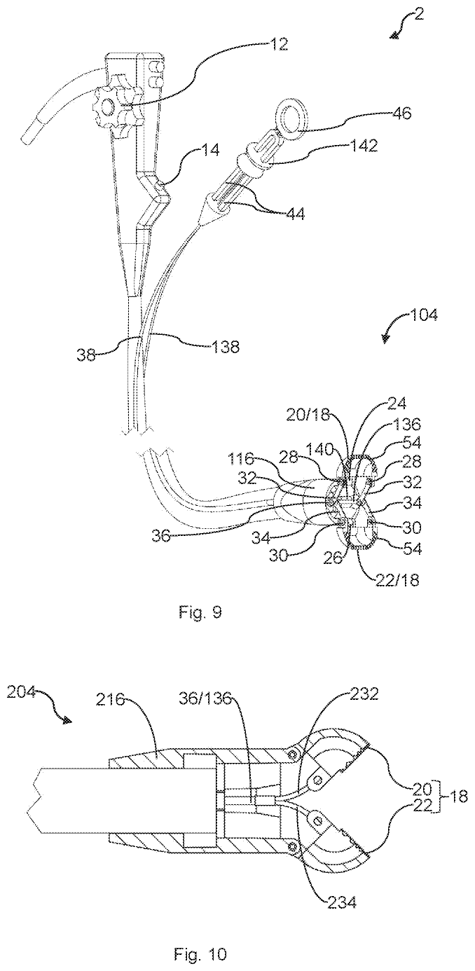

In order to enable stronger gripping as compared to the treatment instrument 4 according to the first embodiment, a treatment instrument 104 according to the invention can additionally be equipped, in accordance with a second embodiment as shown in FIG. 9, with a second actuator 136, a second tube 138 and a second endoscope-external working channel 140. The second actuator 136 is connected to the gripping parts 20 and 22 in accordance with the (first) actuator 36 via the connecting rods 32 and 34. The connecting rods 32 and 34 of the second actuator 136 are pivotally connected to the gripping parts 20 and 22 via joints 28 and 30. The second actuator 136 and, respectively, the distal end thereof, the connecting rods 32 and 34 as well as the joints 28 and 30 of the second actuator 136 and the second working channel 140 and, respectively, the distal end thereof are disposed on a side of the hollow body 116 that is opposed to a side of the hollow body 116 on which the (first) actuator 36 and, respectively, the distal end thereof, the connecting rods 32 and 34 as well as the joints 28 and 30 of the (first) actuator 36 and the (first) working channel 40 and, respectively, the distal end thereof are disposed.

The (first) tube 38 and the second tube 138 are connected to each other at their proximal ends. The (first) actuator 36 and the second actuator 136 are connected to each other at their proximal ends and can be operated via a slide 142 in accordance with the first embodiment.

According to the first two embodiments, the gripping parts 20 and 22 are connected to the respective actuator 36 and/or 136 via rigid connecting rods 32 and 34. As an alternative, in accordance with a third embodiment as shown in FIG. 10, the connection between the actuator 36 and/or 136 and the gripping parts 20 and/or 22 can be made using flexible elements 232 and/or 234. Said flexible elements 232 and/or 234 can be connected to the actuator 36 and/or 136 in a strong or flexurally strong manner and/or can be connected to the corresponding gripping part 20 and/or 22 in a strong or flexurally strong manner so that the joints 28 and 30 can be saved.

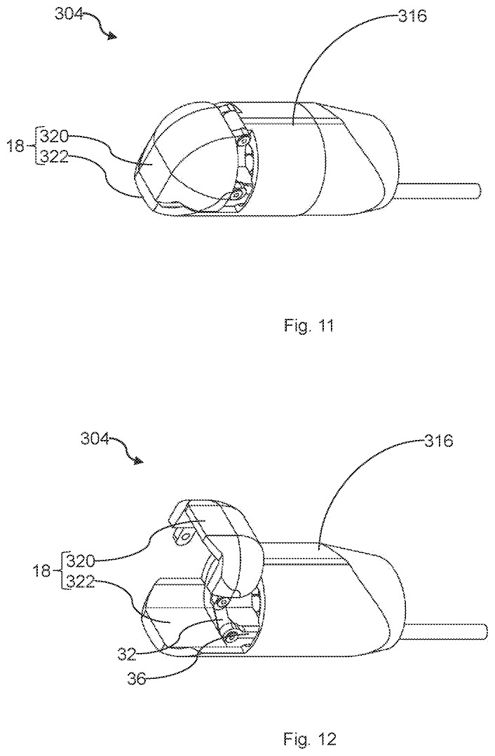

Each of the treatment instruments 4, 104 and 204 according to the invention as set forth in the first, second and third embodiments comprises two gripping parts 20 and 22 both of which are pivotally connected to a hollow body 16, 116 and/or 216. In accordance with a fourth embodiment, a treatment instrument 304 according to the invention may also have two gripping parts 320 and 322 only one (the gripping part 320) of which is pivotally connected to a hollow body 316, whereas the other (the gripping part 322) is connected to the hollow body 316 in a strong or flexurally strong manner. As in the case of the treatment instruments 4, 104 and 204 according to the first, second and third embodiments, the two gripping parts 320 and 322 jointly form a hood-shaped structure that closes a distal side of the hollow body 316, when the gripping parts 320 and 322 are in a closed position (see FIG. 11). In contrast to the gripping parts 20 and 22, which are identical to each other, the gripping parts 320 and 322 are designed differently from each other. The gripping part 322 firmly connected to the hollow body substantially is a projection protruding in the distal direction or a groove-shaped tongue protruding in the distal direction. Only the gripping part 320 which is pivotally connected to the hollow body 316 has a shell or blade shape. The actuation mechanism for adjusting the gripping part 320 corresponds to the actuation mechanism for adjusting the gripping parts 20 and 22.

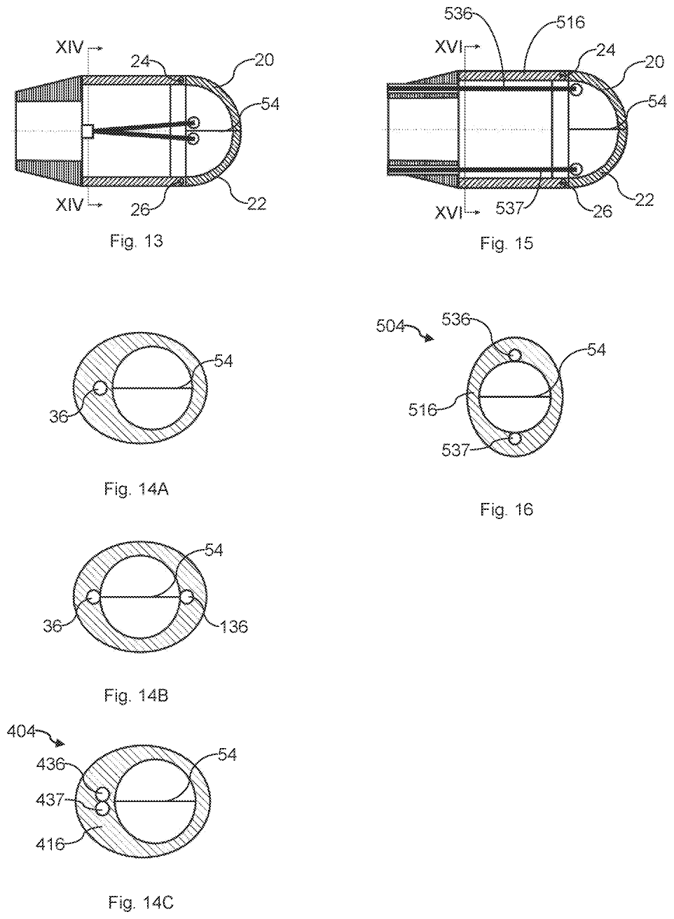

As illustrated by a comparison of the first and second embodiments, a treatment instrument according to the invention may comprise one or two actuators. The FIGS. 14A and 14B illustrating, in accordance with the first and second embodiments, two alternative schematic cross-sectional views along the line XIV-XIV shown in FIG. 13 illustrate the arrangement of the actuators 36 and 136. In accordance with the first embodiment, one single actuator 36 may be movably supported in the hollow body 16 such that the distal end of the actuator 36 extends together with the gripping strips 54 in joint plane, when the gripping parts 20 and 22 are in the closed position. In accordance with the second embodiment, two actuators 36 and 136 may be movably supported in the hollow body 116 such that the distal ends of the two actuators 36 and 136 extend together with the gripping strips 54 in a joint plane, when the gripping parts 20 and 22 are in the closed position. To enable this, the two actuators 36 and 136 according to the second embodiment, or the distal ends thereof, are arranged on opposite sides of the hollow body 116.

In accordance with a fifth and a sixth embodiment, it is also possible, however, to equip a treatment instrument 404 and/or 504 according to the invention with two actuators 436 and 437 and/or 536 and 537 which do not extend together with the gripping strips 54 in a joint plane, or the distal ends of which do not extend together with the gripping strips 54 in a joint plane, when the gripping parts 20 and 22 are in the closed position.

As illustrated in FIG. 14C, the treatment instrument 404 according to the fifth embodiment is equipped with an actuator 436 that is only connected to the gripping part 20 and with an actuator 437 that is only connected to the gripping part 22. The two actuators 436 and 437, or the distal ends thereof, are arranged on a joint side of the hollow body 416 so that, in the closed position of the gripping parts 20 and 22, they are directly adjacent to a plane spanned by the gripping strips 54.

As illustrated by FIG. 15 and FIG. 16 (a cross-sectional view along the line XVI-XVI shown in FIG. 15), the treatment instrument 504 according to the sixth embodiment is equipped with an actuator 536 that is only connected to the gripping part 20 and with an actuator 537 that is only connected to the gripping part 22. The two actuators 536 and 537, or the distal ends thereof, are arranged on opposite sides of the hollow body 516 such that they extend in a joint plane which is perpendicular to the pivot axes of the joints 24 and 26.

Driving of the gripping parts 20 and/or 22 can be refined by providing two actuators 436 and 437 and/or 536 and 537 each of which are only connected to one gripping part 20 or 22.

The arrangement of the actuators 536 and 537 according to the sixth embodiment enables the actuators 536 and 537 to be connected to the corresponding gripping part without any intermediate parts (such as connecting rods, push rods or flexible elements).

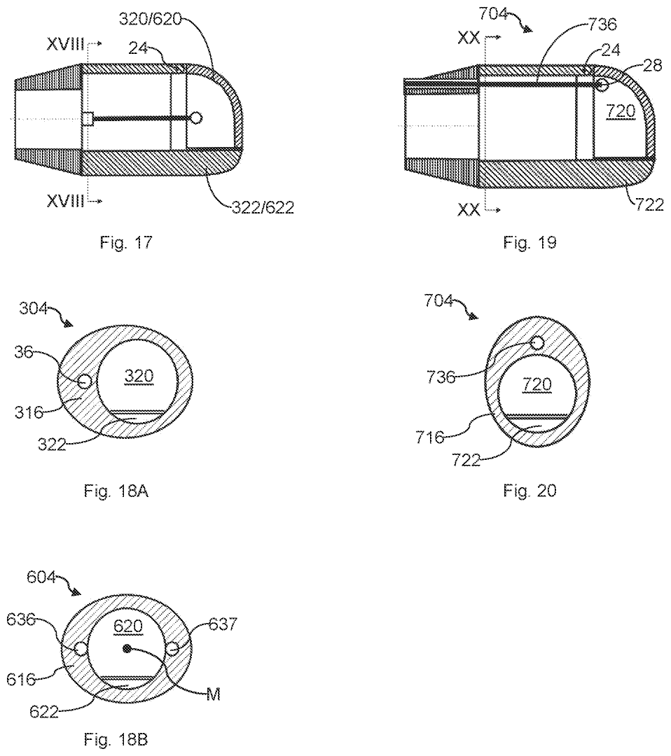

FIGS. 17, 18A, 18B, 19 and 20 show different variations of the arrangement of one actuator or several actuators or of the arrangement of a distal end of one actuator or several distal ends of several actuators in a treatment instrument according to the invention comprising only one gripping part pivotally connected to the hollow body.

FIG. 18A is a schematic cross-sectional view along the line XVIII-XVIII shown in FIG. 17 and illustrates the arrangement of the actuator 36 in the treatment instrument 304 according to the fourth embodiment shown in FIGS. 11 and 12. FIG. 18B is a schematic cross-sectional view along the line XVIII-XVIII shown in FIG. 17 and illustrates a treatment instrument 604 according to a seventh embodiment comprising an actuator 636 and a second actuator 637. The two actuators 636 and 637, or the distal ends of the two actuators 636 and 637, are disposed on two opposite sides of the hollow body 616. The two actuators 636 and 637 or the distal ends of the two actuators 636 and 637, are located in a joint plane that extends across a longitudinal central axis M of the hollow body 616.

As FIG. 19 and FIG. 20 (a cross-sectional view along the line XX-XX shown in FIG. 19) illustrate, according to an eighth embodiment an actuator 736 can be arranged in a treatment instrument 704 comprising merely one gripping part 720 pivotally connected to a hollow body 716 such that a joint 28 between the actuator 736 and the gripping part 720 is directly adjacent to a joint 24 between the gripping part 720 and the hollow body 716. Just as in the treatment instrument 504 according to the sixth embodiment, this arrangement of the actuator 736 can enable a direct connection between the actuator 736 and the gripping part 720 without any intermediate parts (such as connecting rods, push rods or flexible elements).

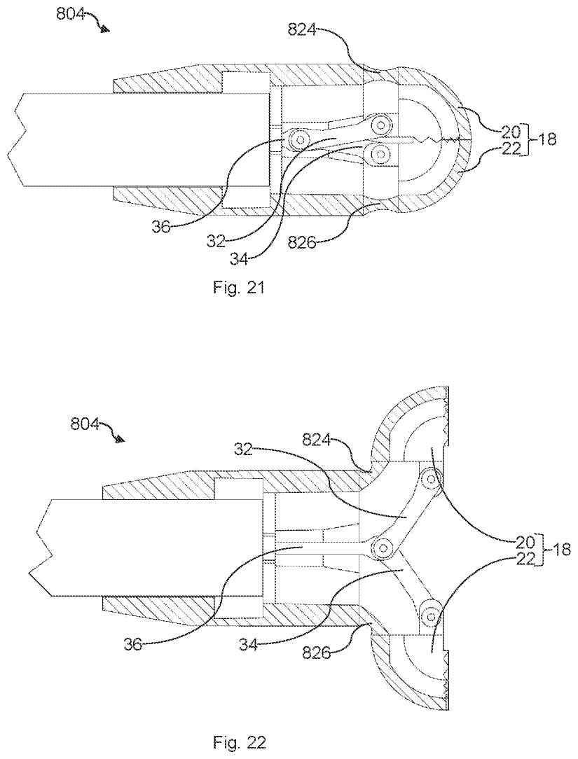

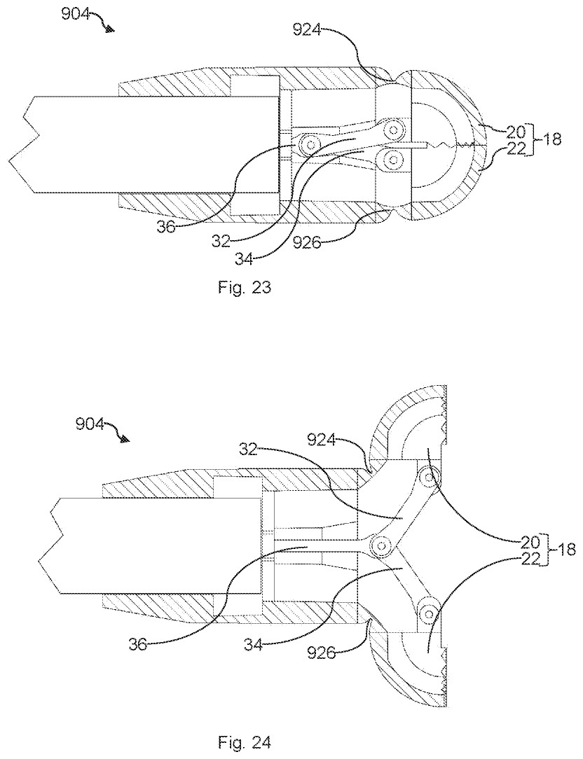

FIG. 21 and FIG. 22 illustrate a treatment instrument 804 according to a ninth embodiment. FIG. 23 and FIG. 24 illustrate a treatment instrument 904 according to a tenth embodiment.

The treatment instrument 804 in accordance with the ninth embodiment differs from the treatment instrument 4 in accordance with the first embodiment merely insofar as the gripping parts 20 and 22 are connected to the hollow body 16 not via swivel joints 24 and 26 but via solid-body joints 824 and 826.

The treatment instrument 904 in accordance with the tenth embodiment differs from the treatment instrument 4 in accordance with the first embodiment merely insofar as the gripping parts 20 and 22 are connected to the hollow body 16 not via swivel joints 24 and 26 but via film hinges 924 and 926.

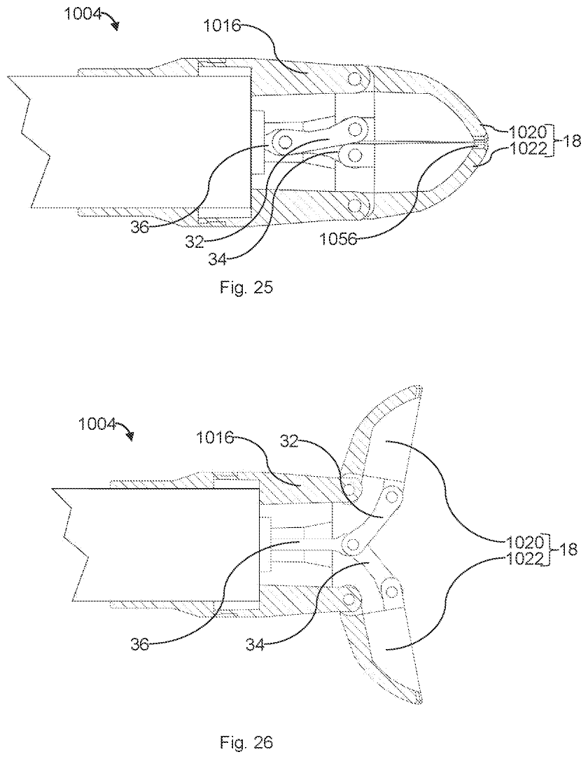

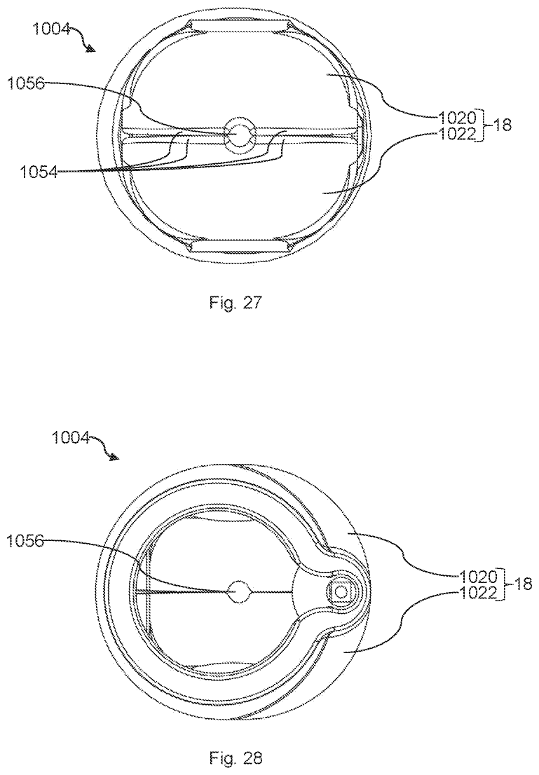

FIG. 25, FIG. 26, FIG. 27 and FIG. 28 show a treatment instrument 1004 in accordance with an eleventh embodiment. The treatment instrument 1004 according to the eleventh embodiment differs from the treatment instrument 4 according to the first embodiment as regards two aspects.

On the one hand, the gripping parts 1020 and 1022 of the treatment instrument 1004 are configured to together form, in a closed position (as shown in FIG. 25), a hood-shaped structure which does not only close the hollow body 1016 in the distal direction, but at the same time also forms a tapered shape. In other words, the gripping parts 1020 and 1022 are configured to have or form, in the closed position, a portion conically widening in the proximal direction. In addition to the screening from impurities, this geometric shape makes it possible to widen and to pass strictures, if necessary. Apart from the passive widening by the conical shape, the gripping parts 1020 and 1022 can actively exert pressure on the stricture by opening and can assist the widening process. In a distal portion, also the hollow body 1016 comprises a portion conically widening in the proximal direction.

On the other hand, the gripping parts 1020 and 1022 according to the twelfth embodiment differ from the gripping parts 20 and 22 according to the first embodiment as regards a recess 1056. The recess 1056 is configured partially in the gripping part 1020 and partially in the gripping part 1022. More precisely, gripping strips 1054 of each of the gripping parts 1020 and 1022 are centrally interrupted by a groove-shaped portion. When the gripping parts 1020 and 1022 are in the closed position, the groove-shaped portions of the two gripping parts 1020 and 1022 are adjacent to each other and jointly form the recess 1056. The recess includes a circular contour. The opening width and, respectively, the diameter of the recess 1056 amounts to 1.1 mm. The recess 1056 enables a guide wire to be put through the closed gripping element without unduly abandoning the protection of a lens of an optical system of an endoscope. In particular, the recess 1056 is arranged and a corresponding endoscope 2 is formed such that the gripping element 18 in the closed position merely uncovers a guide channel for the guide wire in the distal direction and completely covers all remaining functional units 8 such as an outlet of a primary endoscope-internal working channel, an optical system for imaging, lamps and/or a rinsing channel outlet opening of a rinsing channel in the distal direction.

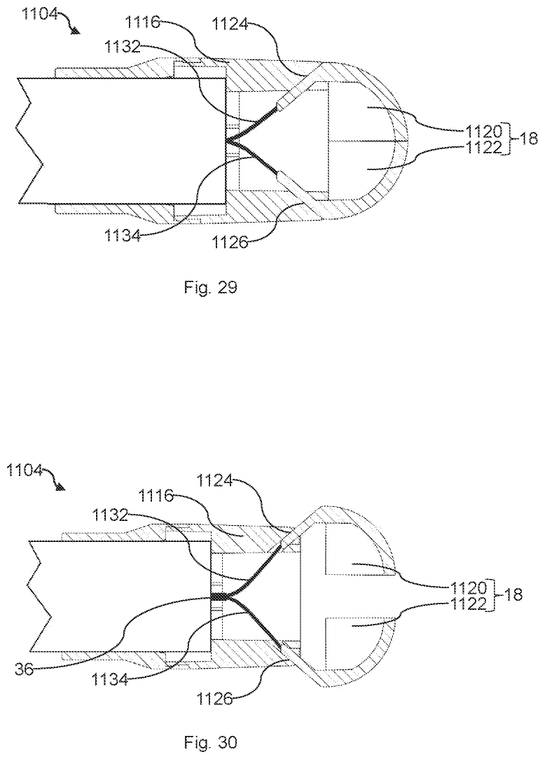

FIG. 29 and FIG. 30 illustrate a treatment instrument 1104 according to a twelfth embodiment. The treatment instrument 1104 according to the twelfth embodiment differs from the treatment instrument 4 according to the first embodiment insofar as the gripping parts 1120 and 1122 are connected to a hollow body 1116 not via swivel joints 24 and 26 but via slider joints 1124 and 1126. The slider joints 1124 and 1126 are configured such that the gripping parts 1120 and 1122 are moved outwards both in the distal direction and in a radial direction, when the gripping parts 1124 and 1126 are brought into an opened position (see FIG. 30) via flexible elements 1132 and 1134 strongly connected to the gripping parts 1124 and 1126, and that the gripping parts 1120 and 1122 are moved inwards both in the proximal direction and in a radial direction, when the gripping parts 1124 and 1126 are brought into a closed position (see FIG. 29) via the flexible elements 1132 and 1134.

In other words, in the treatment instrument 1104 according to the twelfth embodiment, the gripping element 18 is in the form of a parallel gripper.

### LIST OF REFERENCE NUMERALS

2 endoscope
4 treatment instrument according to the first embodiment
6 endoscope head
8 functional unit inside the endoscope head
10 shaft of the endoscope
12 handle of the endoscope
14 inlet of the endoscope-internal working channel
16 hollow body
18 gripping element/gripping unit
20, 22 gripping part/gripping branch
24, 26 joint between hollow body and gripping part
28, 30 joint between gripping part and connecting rod
32, 34 connecting rod
36 actuator
38 tube
40 endoscope-external working channel
42 slide
44 guide rail
46 ring at guide rails
48 recess at the proximal end of the hollow body
50 stop
52 recess at the distal end of the hollow body
54 gripping strip/branch
104 treatment instrument according to the second embodiment
116 hollow body according to the second embodiment
136 second actuator
138 second tube
140 second endoscope-external working channel
142 slide according to the second embodiment
204 treatment instrument according to the third embodiment
216 hollow body according to the third embodiment
232, 234 flexible element
304 treatment instrument according to the fourth embodiment
316 hollow body according to the fourth embodiment
320 pivotal gripping part/gripping branch
322 fixed gripping part
404 treatment instrument according to the fifth embodiment
416 hollow body according to the fifth embodiment
436, 437 actuator according to the fifth embodiment
504 treatment instrument according to the sixth embodiment
516 hollow body according to the sixth embodiment
536, 537 actuator according to the sixth embodiment
604 treatment instrument according to the seventh embodiment
616 hollow body according to the seventh embodiment
620 pivotal gripping part/branch according to the seventh embodiment
622 fixed gripping part according to the seventh embodiment
636, 637 actuator according to the seventh embodiment
704 treatment instrument according to the eighth embodiment
716 hollow body according to the eighth embodiment
720 pivotal gripping part according to the eighth embodiment
722 fixed gripping part according to the eighth embodiment
736 actuator according to the eighth embodiment
804 treatment instrument according to the ninth embodiment
824, 826 solid-body joint
904 treatment instrument according to the tenth embodiment
924, 926 film hinge
1004 treatment instrument according to the eleventh embodiment
1016 hollow body according to the eleventh embodiment
1020, 1022 gripping part according to the eleventh embodiment
1054 gripping strip according to the eleventh embodiment
1056 recess in gripping part
1104 treatment instrument according to the twelfth embodiment
1116 hollow body according to the twelfth embodiment
1120, 1122 gripping part according to the twelfth embodiment
1124, 1126 slider joint
1132, 1134 flexible element according to the twelfth embodiment

1-16. (canceled)

17. A medical treatment instrument which is provided and configured to be coupled to a distal end or end portion of a medical endoscope to become an adaptive member of the medical endoscope, the medical treatment instrument comprising:
a sleeve-shaped hollow body comprising a circumferential wall which internally surrounds an axially extending cavity being open into distal direction and comprising a proximal, plug-on-like coupling portion, that is provided and configured to be attached or fitted to or onto an endoscope shaft or endoscope head of the endoscope,
a gripping unit that is supported on or articulated to a distal circumferential wall portion of the hollow body and
an actuation mechanism or transmission provided for operating the gripping unit at least from a release position to a gripping position, said actuation mechanism or transmission having a proximal portion that extends from the hollow body to a proximal direction, and having a distal portion that is received in the cavity and comprises a distal end or end portion which is coupled or connected to the gripping unit at an inner side thereof facing the cavity, wherein
the gripping unit, when in the gripping position, takes the shape of a cap, flap, cupola, door or roof by which the cavity is closed at a distal end of the hollow body at least when the gripping position is reached.

18. The medical treatment instrument according to claim 17, wherein the gripping unit is formed to be transparent at least in portions.

19. The medical treatment instrument according to claim 17, wherein the gripping unit comprises only one gripping branch or two gripping branches articulated to the distal circumferential wall portion of the hollow body and being coupled or connected with the actuation mechanism or transmission

20. The medical treatment instrument according to claim 17, wherein the only one gripping branch or the two gripping branches comprises, a flat plate portion, a single-curved shell portion or a shell portion conically widening radially outwards in the longitudinal direction of the hollow body which is arranged, in a closed position of the gripping element (18), on a distal end side of the hollow body.

21. The medical treatment instrument according to claim 20, wherein an edge portion of a proximal end of the only one gripping branch or the two gripping branches is connected to an edge portion of a distal end of the hollow body via a joint to be pivoting about a swivel axis such that the swivel axis extends tangentially to both portions.

22. The medical treatment instrument according to claim 19, wherein the actuation mechanism or transmission comprises at least one actuator in the form of a cable movably supported in the hollow body which is connected to the gripping element especially the only one gripping branch or the two gripping branches via at least one connecting rod pivotally connected to each of the actuator and the gripping element or via at least one flexible element strongly connected to each of the actuator and the gripping element, and the gripping element can be operated by driving the actuator.

23. The medical treatment instrument according to claim 22, wherein the connecting rod is connected to be pivoting about a swivel axis to a further portion of the proximal end edge of the only one gripping branch or the two gripping branches via a joint such that the swivel axis is perpendicular to the further portion.

24. The medical treatment instrument according to claim 22, wherein:
the gripping element comprises two gripping parts,
one of the two gripping parts is firmly connected to the hollow body,
the other of the two gripping parts is the only one gripping branch being pivotally or slidingly connected to the hollow body and
the at least one connecting rod or the at least one flexible element connects the gripping branch that is pivotally or slidingly connected to the hollow body to the actuator.

25. The medical treatment instrument according to claim 24, wherein:
the medical treatment instrument comprises a further actuator,
a further connecting rod or a further flexible element connects the gripping branch that is pivotally or slidingly connected to the hollow body to the further actuator, and
a distal end of the actuator and a distal end of the further actuator are arranged on two opposite sides of the hollow body.

26. The medical treatment instrument according to claim 22, wherein:
the gripping element comprises the two gripping branches which are pivotally or slidingly connected to the hollow body,
the at least one connecting rod or the at least one flexible element connects the actuator to one of the two gripping branches, and
a further connecting rod or a further flexible element connects the actuator to the other of the two gripping branches.

27. The medical treatment instrument according to claim 26, wherein:
the treatment medical instrument comprises a further actuator,
the further actuator is connected to each of the two gripping branches via a further connecting rod or a further flexible element, and
a distal end of the actuator and a distal end of the further actuator are arranged on two opposite sides of the hollow body.

28. The medical treatment instrument according to claim 22, wherein:
the gripping element comprises the two gripping branches which are pivotally or slidingly connected to the hollow body,
the at least one connecting rod or the at least one flexible element connects the actuator to one of the two gripping branches,
the medical treatment instrument comprises a further actuator and a further connecting rod or a further flexible element, and
the further connecting rod or the further flexible element connects the further actuator to the other of the two gripping branches.

29. The medical treatment instrument according to claim 26, wherein the two gripping branches are arranged to be opposed to each other so that the actuator and/or the further actuator extend(s) symmetrically to/on/in a plane situated centrally between the two gripping branches in the closed condition.

30. The medical treatment instrument according to claim 17, wherein the gripping element comprises a recess with a maximum opening width of 1.1 mm or less.

31. A medical endoscope having a distal end or head-like end portion which is coupled to a medical treatment instrument according to claim 17, wherein a proximal portion of the actuation mechanism or transmission in the form of a cable of the medical treatment instrument is guided along an outer side of the endoscope.

32. A medical endoscope having a distal end which comprises a lens of an optical system on an end face and which is coupled to a medical treatment instrument, wherein the medical treatment instrument comprises a hollow body and a gripping unit, the gripping unit is operable via an actuation mechanism or transmission which comprises a distal end or end portion situated inside the hollow body, which extends from the hollow body to a proximal direction and which comprises a proximal portion that can be guided along an outer side of the endoscope;
wherein the gripping unit is configured in the form of a cap which, in a condition of the medical treatment instrument attached to the endoscope, can be brought into a closed position in which the lens of the optical system of the endoscope is covered by the gripping unit in the distal direction.

33. The medical endoscope according to claim 31, characterized in that the endoscope comprises a rinsing channel through which rinsing liquid can be conveyed to a rinsing channel outlet opening disposed on the end face of the endoscope, in the condition of the medical treatment instrument attached to the endoscope the gripping unit is covered at least partially in the distal direction in the closed position of the rinsing channel outlet opening and an inner face of the gripping unit facing in the proximal direction is spaced apart from the lens of the optical system and from the rinsing channel outlet surface in the closed position of the gripping unit such that, when rinsing liquid is conveyed to the rinsing channel outlet opening, rinsing liquid impinges on the inner face of the gripping unit facing the proximal direction after exiting the rinsing channel outlet opening and is reflected to the lens of the optical system.

34. The medical endoscope according to claim 32, characterized in that the endoscope comprises a rinsing channel through which rinsing liquid can be conveyed to a rinsing channel outlet opening disposed on the end face of the endoscope, in the condition of the medical treatment instrument attached to the endoscope the gripping unit is covered at least partially in the distal direction in the closed position of the rinsing channel outlet opening and an inner face of the gripping unit facing in the proximal direction is spaced apart from the lens of the optical system and from the rinsing channel outlet surface in the closed position of the gripping unit such that, when rinsing liquid is conveyed to the rinsing channel outlet opening, rinsing liquid impinges on the inner face of the gripping unit facing the proximal direction after exiting the rinsing channel outlet opening and is reflected to the lens of the optical system.
